(19) United States
(12) Patent Application Publication
(10) Pub. No.: US 2008/0146664 A1
(43) Pub. Date: Jun. 19, 2008

(54) Method for treating diseases caused by accumulation of body fat

(76) Inventors: Hitoshi Okuyama, Nagoya-Shi (JP); Yoshihiro Kawabata, Tokyo-To (JP); Kenichi Katayama, Tokyo-To (JP); Osamu Ezaki, Tokyo-To (JP); Nobuyo Kasaoka, Saitama-shi (JP)

Correspondence Address: WENDEROTH, LIND & PONACK, L.L.P., 2033 K STREET N. W., SUITE 800, WASHINGTON, DC, 20006-1021, US

(21) Appl. No.: 11/705,718

(22) Filed: Feb. 14, 2007

(30) Foreign Application Priority Data
Dec. 15, 2006 (JP) 2006-338598

(51) Int. Cl. A61K 31/202; A61K 31/22; A61P 3/06; A61K 36/185

(52) U.S. Cl. 514/549; 514/560

(57) ABSTRACT

According to the present invention, there is provided a method of treating diseases caused by the accumulation of body fat, comprising the step of administering a therapeutically effective amount of punicic acid or its ester to a mammal or fish.

### BACKGROUND OF THE INVENTION

1. Field of the Invention

The present invention relates to a method for treating diseases caused by the accumulation of body fat. More specifically, the present invention relates to a method for treating diseases caused by the accumulation of body fat, comprising the step of administering a therapeutically effective amount of punicic acid or its ester to a mammal or fish.

2. Background Technology

In recent years, even in Japan, due to a change in dietary habits and a lack of exercise, the number of obese patients has increased, which has become an object of great public concern. Obesity can be defined as an excessive accumulation of body fat. In particular, the accumulation of visceral fat causes lifestyle-related diseases such as diabetes and hyperlipidemia, so that the development of medicines and foods to prevent obesity has been desired.

To date, it has been reported that processed fats and oils for food products comprising conjugated highly-unsaturated fatty acid glycerides having a conjugated triene structure are effective in controlling and reducing the accumulation of perirenal adipose tissue and periovular adipose tissue (Japanese Patent Laid-open No. 2002-165559). However, there have been no suggestions or disclosures regarding the effectiveness of fats and oils comprising conjugated highly-unsaturated fatty acids having a conjugated triene structure in controlling and reducing the weight of the liver and the accumulation of subcutaneous fat.

Further, to date, it has been reported that a diet supplemented with pomegranate seed oil has no effect in controlling the weight of white adipose tissue in the abdomen and serum lipid levels (Arao, K., et al. (2004) Bio Med Central 3(24)) and that serum lipid levels in mice fed a diet supplemented with pomegranate seed oil increases as compared to that in a control group and the supplementation has no effect in controlling the weight of adipose tissue (Yamasaki, M., et al. (2006) Nutrition 22: 54-59). On the other hand, it has been reported that by adding pomegranate seed oil, weight gain is controlled (Japanese Patent Laid-open No. 2005-179264) and the weight of perirenal adipose tissue tends to be controlled (Japanese Patent Laid-open No. 2005-239704). However, it has not been known that pomegranate seed oil which is rich in punicic acid is effective in controlling the weight of the liver and the accumulation of subcutaneous fat.

### SUMMARY OF THE INVENTION

The present inventors have found that the accumulation of body fat is controlled by replacing safflower oil contained in a high carbohydrate diet with pomegranate seed oil (Example 2). The present inventors have also found that the accumulation of body fat, more specifically visceral fat such as parametrial adipose tissue and retroperitoneal adipose tissue and subcutaneous fat is significantly controlled by partially replacing safflower oil contained in a high fat diet with pomegranate seed oil (Example 2). The present inventors have further found that the increase in the weight of the liver is significantly controlled by partially replacing safflower oil contained in a high fat diet with pomegranate seed oil (Example 2). The present invention is based on these findings.

An objective of the present invention is to provide a method of treating diseases caused by the accumulation of body fat and a method of reducing the accumulation of body fat.

Further, an objective of the present invention is to provide a therapeutic agent for diseases caused by the accumulation of body fat, a food additive for use in reducing the accumulation of body fat, and a body fat accumulation-reducing agent.

According to the present invention, there is provided a method of treating diseases caused by the accumulation of body fat, comprising the step of administering a therapeutically effective amount of punicic acid or its ester to a mammal or fish.

According to the present invention, there is provided a method of reducing the accumulation of body fat, comprising the step of administering an effective amount of punicic acid or its ester to a mammal or fish.

According to the present invention, there is provided a therapeutic agent for diseases caused by the accumulation of body fat, comprising punicic acid or its ester as an active ingredient.

According to the present invention, there is provided a food additive for use in reducing the accumulation of body fat, comprising punicic acid or its ester.

According to the present invention, there is provided a body fat accumulation-reducing agent, comprising punicic acid or its ester.

According to the present invention, in the case where pomegranate seed oil is used as an active ingredient, diseases caused by the accumulation of body fat can be treated by changing the kind of oil without changing the amount of fat intake, so that an effective diet therapy without lowering the quality of life can be advantageously provided.

Further, the present invention is advantageous from the viewpoint of utilization of waste materials since pomegranate seeds have been largely discarded upon manufacturing pomegranate juice products. Further, punicic acid in pomegranate seed oil is advantageously present as a triglyceride which has a lower acid value and a better flavor than punicic acid as a fatty acid.

### BRIEF DESCRIPTION OF THE DRAWINGS

FIG. 1 shows the change in the amount of food intake in individual groups in Example 1.

FIG. 2 shows the average amount of food intake (for 4 days) in individual groups in Example 1.

FIG. 3 shows the change in body weight in individual groups in Example 1.

FIG. 4 shows the final body weight in individual groups in Example 1.

FIG. 5 shows the weight of parametrial adipose tissue in individual groups in Example 1.

FIG. 6 shows the weight of retroperitoneal adipose tissue in individual groups in Example 1.

FIG. 7 shows the weight of the liver in individual groups in Example 1.

FIG. 8 shows the change in body weight in individual groups in Example 2.

FIG. 9 shows the final body weight in individual groups in Example 2.

FIG. 10 shows the weight of body fat in individual groups in Example 2.

FIG. 11 shows body fat percentage in individual groups in Example 2.

FIG. 12 shows the weight of the liver in individual groups in Example 2.

FIG. 13 shows the weight of parametrial adipose tissue in individual groups in Example 2.

FIG. 14 shows the weight of brown adipose tissue in individual group in Example 2.

FIG. 15 shows the weight of retroperitoneal adipose tissue in individual groups in Example 2.

FIG. 16 shows the weight of subcutaneous adipose tissue in individual groups in Example 2.

### DETAILED DESCRIPTION OF THE INVENTION

The present invention will be explained in more details as follows. The following description is presented for the purpose of illustrating the present invention and is not intended to limit the invention to the described embodiments only. The present invention can be carried out in various forms as long as they do not depart from the spirit and scope of the invention.

Prior art literature and patent literature such as Patent Application Publication Gazette cited in the present specification are all incorporated into this specification by reference and can be used to carry out the present invention.

### Active Ingredients

Punicic acid used as an active ingredient in the present invention is a kind of conjugated linolenic acid.

Punicic acid is contained in pomegranate seed oil and the like usually at about 30 to 90%.

An ester of punicic acid used as an active ingredient in the present invention is, for example, a glyceride comprising punicic acid as a constituent fatty acid. Examples of the glyceride include monoglycerides, diglycerides and triglycerides; however, triglycerides are preferred.

Punicic acid or its ester used as an active ingredient in the present invention can be obtained commercially.

Punicic acid or its ester used as an active ingredient in the present invention can be manufactured according to a known method. For example, punicic acid used as an active ingredient in the present invention can be manufactured according to a method described in “Edible Oils and Their Production” (1992) by Saiwai Shobo. Further, an ester of punicic acid used as an active ingredient in the present invention can be manufactured according to a method described in “Chemical Industry of Fat and Oil” (1961) by Kogyo Gijutsu Shinsho (New Industrial Technology).

Punicic acid or its ester used as an active ingredient in the present invention can be purified from pomegranate seed oil.

Punicic acid or its ester used as an active ingredient in the present invention is preferably obtained from pomegranate seed oil; pomegranate seed oil can also be used as it is as an active ingredient.

Pomegranate seed oil can be obtained commercially or manufactured according to a known method.

In the present invention, when punicic acid or its ester is used as a therapeutic agent, the punicic acid or its ester content in the therapeutic agent is preferably higher than 2.5% by weight, more preferably 2.5 to 90% by weight.

### Use

According to Example 2, decreasing tendencies in the weight of body fat and body fat percentage were observed in a group fed a high carbohydrate diet (low fat diet) supplemented with pomegranate seed oil. Further, decreasing tendencies in parametrial adipose tissue and retroperitoneal adipose tissue were observed. Furthermore a decreasing tendency in subcutaneous adipose tissue was observed.

Also according to Example 2, it was observed that the weight of body fat and the body fat percentage were significantly decreased in a group fed a high fat diet supplemented with pomegranate seed oil, and further the body weight was significantly increased in a group fed a high fat diet from 12 weeks after the start of experimental feeding, while it was controlled in a group fed a high fat diet supplemented with pomegranate seed oil. Further, the weights of the liver, parametrial adipose tissue, and retroperitoneal adipose tissue were significantly decreased. Furthermore, a significant decrease in the weight of subcutaneous adipose tissue was observed.

From the results above, it has been recognized that the weight of body fat, specifically the weights of visceral fat and subcutaneous fat can be significantly decreased by the administration of pomegranate seed oil. This demonstrates that the accumulation of body fat can be reduced by the addition of punicic acid or its ester or pomegranate seed oil containing them preferably to a high fat diet, whereby diseases caused by the accumulation of body fat, specifically diseases caused by the accumulation of visceral fat or the diseases caused by the accumulation of subcutaneous fat, can be improved. Accordingly, punicic acid or its ester or pomegranate seed oil containing them can be used for the treatment of diseases caused by the accumulation of visceral fat or diseases caused by the accumulation of subcutaneous fat. In particular, since a significant decrease in the weight of the liver and a significant decrease in parametrial adipose tissue were observed, punicic acid or its ester or pomegranate seed oil containing them can be used for the treatment of fatty liver or visceral fat-type obesity. Further, since a significant decrease in subcutaneous adipose tissue was observed, punicic acid or its ester or pomegranate seed oil containing them can be used for the treatment of subcutaneous fat-type obesity.

The present invention is advantageous in that lipodystrophy-like symptoms (fatty liver, splenic hypertrophy, and disappearance of brown fat), which are generated by the addition of conjugated linoleic acid (Tsuboyama-Kasaoka, N., et al. (2003) J. Nutr. 133: 1793-1799), are not observed. Further, the present invention is advantageous in that a partial decrease in adipose tissue, which is observed by the addition of conjugated linoleic acid, is not observed and all adipose tissue decreases to the same degree.

The term “treatment” as used in the present specification includes prevention and improvement.

In the present specification, “body fat” includes visceral fat and subcutaneous fat.

In the present specification, “visceral fat” include hepatic fat and parametrial fat, preferably hepatic fat.

In the present specification, “diseases caused by the accumulation of body fat” include diseases caused by the accumulation of visceral fat and diseases caused by the accumulation of subcutaneous fat.

In the present specification, “diseases caused by the accumulation of visceral fat” include fatty liver and visceral fat-type obesity, preferably fatty liver.

In the present specification, “diseases caused by the accumulation of subcutaneous fat” include subcutaneous fat-type obesity.

An example of a preferred embodiment of a therapeutic method according to the present invention is a method of treating fatty liver, comprising the step of administering a therapeutically effective amount of punicic acid or its triglyceride to a mammal or fish.

An example of a preferred embodiment of a therapeutic method according to the present invention is also a method of treating diseases caused by the accumulation of subcutaneous fat (preferably subcutaneous fat-type obesity), comprising the step of administering a therapeutically effective amount of punicic acid or its triglyceride to a mammal or fish.

An example of a more preferred embodiment of a therapeutic method according to the present invention is a method of treating fatty liver, comprising the step of administering a therapeutically effective amount of punicic acid obtained from pomegranate seed oil or its triglyceride to a mammal or fish.

An example of a more preferred embodiment of a therapeutic method according to the present invention is a method of treating diseases caused by the accumulation of subcutaneous fat (preferably subcutaneous-type obesity), comprising the step of administering a therapeutically effective amount of punicic acid obtained from pomegranate seed oil or its triglyceride to a mammal or fish.

An example of a particularly preferred embodiment of a therapeutic method according to the present invention is a method of treating fatty liver, comprising the step of administering to a mammal or fish 2.5 to 90% by weight of punicic acid obtained from pomegranate seed oil or its triglyceride.

An example of a particularly preferred embodiment of a therapeutic method according to the present invention is further a method of treating diseases caused by the accumulation of subcutaneous fat (preferably subcutaneous-type obesity), comprising the step of administering to a mammal or fish 2.5 to 90% by weight of punicic acid obtained from pomegranate seed oil or its triglyceride.

An example of a preferred embodiment of a reducing method according to the present invention is a method of reducing the accumulation of hepatic fat, comprising the step of administering an effective amount of punicic acid or its triglyceride to a mammal or fish.

An example of a preferred embodiment of a reducing method according to the present invention is also a method of reducing the accumulation of subcutaneous fat, comprising the step of administering an effective amount of punicic acid or its triglyceride to a mammal or fish.

An example of a more preferred embodiment of a reducing method according to the present invention is a method of reducing the accumulation of hepatic fat, comprising the step of administering an effective amount of punicic acid obtained from pomegranate seed oil or its triglyceride to a mammal or fish.

An example of a more preferred embodiment of a reducing method according to the present invention is a method of reducing the accumulation of subcutaneous fat, comprising the step of administering an effective amount of punicic acid obtained from pomegranate seed oil or its triglyceride to a mammal or fish.

An example of a particularly preferred embodiment of a reducing method according to the present invention is a method of reducing the accumulation of hepatic fat, comprising the step of administering to a mammal or fish 2.5 to 90% by weight of punicic acid obtained from pomegranate seed oil or its triglyceride.

An example of a particularly preferred embodiment of a reducing method according to the present invention is further a method of reducing the accumulation of subcutaneous fat, comprising the step of administering to a mammal or fish 2.5 to 90% by weight of punicic acid obtained from pomegranate seed oil or its triglyceride.

A therapeutic agent, a food additive, and a body fat accumulation-reducing agent according to the present invention can be manufactured by using punicic acid or its ester or pomegranate seed oil containing them as an active ingredient and mixing with pharmaceutically acceptable carriers, excipients, binding agents, diluting agents, and the like. Carriers, excipients, binding agents, diluting agents, and the like which can be used in manufacturing medicines and food additives are known and can be appropriately selected depending on the dosage form.

The dose of punicic acid or its ester can be appropriately determined based on various factors, such as the route of administration, the degree of symptoms, and the age, gender, and body weight of the patients. For example, when the dose for mice in examples is converted based on the body weight into the dose for humans, the daily dose for adult humans (body weight: 50 to 60 kg) can be 100 to 120 g for oral administration; however, a designated effect can be expected at a daily dose of 10 to 150 g. Further, when the dose for mice in examples is converted based on the calorie intake into the dose for humans, the daily dose for adult humans (2000 kcal/day intake) can be 10 to 15 g for oral administration; however, a designated effect can also be expected at a daily dose of 1 to 40 g. The actual dose may vary depending on the degree of symptoms and the like and may depart from the abovementioned dose ranges.

In the present invention, when punicic acid or its ester or pomegranate seed oil containing them is added to food products, the kind of food products to which it is added is not particularly limited. Examples of the food products include jelly, drinks such as yogurts, confectionery such as biscuits and fat cakes, fish/meat products such as sausages and wienerwursts, dairy products such as butter, edible oils such as salad oils and frying oils, processed oil and fat food products such as margarines, seasonings such as dressings and mayonnaise.

According to the present invention, there is provided use of punicic acid or its ester or pomegranate seed oil containing them for manufacture of a therapeutic agent for diseases caused by the accumulation of body fat.

According to the present invention, there is also provided use of punicic acid or its ester or pomegranate seed oil containing them for manufacture of a food additive used for reducing the accumulation of body fat.

The term “mammal” in the present specification includes animals kept for companionship such as dogs and cats, livestock such as cattle and pigs, and humans. Further, the term “fish” includes cultured fish.

### EXAMPLES

The following examples will explain the present invention more in details; however, they are not to be construed to limit the scope of the invention.

### Example 1

### Effect of Short-Term Administration of Pomegranate Seed Oil on the Accumulation of Visceral Fat

### 1. Experimental Animals and Experimental Conditions

Female C57BL6j mice at 8 weeks of age (obtained from SLC) were divided into four groups of five animals: (1) a group fed a high carbohydrate diet (hereinafter referred to as “HC diet group”), (2) a group fed a high fat diet (hereinafter referred to as “HF diet group”), (3) a group fed HF diet+5% pomegranate seed oil (hereinafter referred to as “5% pomegranate seed oil-supplemented diet group”), and (4) a group fed HF diet+10% pomegranate seed oil (hereinafter referred to as “10% pomegranate seed oil-supplemented diet group”), fed for 6 days, and then subjected to dissection. The animals were fed ad libitum during the feeding period. The animals were sufficiently fed the day before dissection and the dissection was carried out under non-fasting conditions.

The fatty acid composition of pomegranate seed oil is shown in Table 1

TABLE 1

| Fatty acid | | Content |
|---|---|---|
| C16:0 | Palmitic acid | 3.80% |
| C18:0 | Stearic acid | 2.70% |
| C18:1 c9 | Oleic acid | 7.20% |
| C18:2 c9, c12 | Linoleic acid | 8.60% |
| C18:3 c9, t11, | Punicic acid | 77.70% |
| c13 | (conjugated linolenic acid, CLNA) | |

The compositions of individual samples administered are shown in Table 2.

TABLE 2

| | High fat diet (g) | |
|---|---|---|
| High | | +Pomegranate |
| carbohydrate | | seed oil |

| | diet (g) | | 5% | 10% |
|---|---|---|---|---|
| Safflower oil | 4.00 | 33.50 | 28.50 | 23.50 |
| Pomegranate | | | 5.00 | 10.00 |
| seed oil | | | | |
| Casein | 20.00 | 29.00 | 29.00 | 29.00 |
| Sucrose | 11.03 | 23.29 | 23.29 | 23.29 |
| a-Starch | 55.17 | 0.00 | 0.00 | 0.00 |
| Vitamin mix | 1.00 | 1.45 | 1.45 | 1.45 |
| (AIN-93) | | | | |
| Mineral mix | 3.50 | 5.08 | 5.08 | 5.08 |
| (AIN-93) | | | | |
| Cellulose | 5.00 | 7.25 | 7.25 | 7.25 |
| L-cystine | 0.30 | 0.44 | 0.44 | 0.44 |
| Total | 100 | 100 | 100 | 100 |

### 2. Measurements

Body weight and the amount of food intake were measured daily during the feeding period.

Parametrial adipose tissue, retroperitoneal adipose tissue, and the liver were taken upon dissection, and weighed.

### 3. Experimental Results

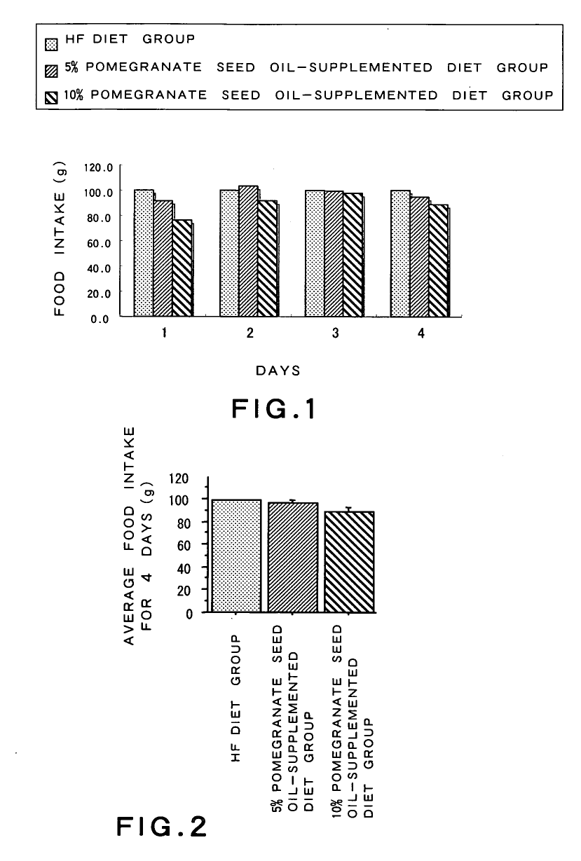

The change in the amount of food intake and the average food intake (for 4 days) in individual groups were shown in FIG. 1 and FIG. 2, respectively. As for the amount of food intake, no decrease was observed in the group fed the 5% pomegranate seed oil-supplemented diet, while a slight decrease was observed in the group fed the 10% pomegranate seed oil-supplemented diet.

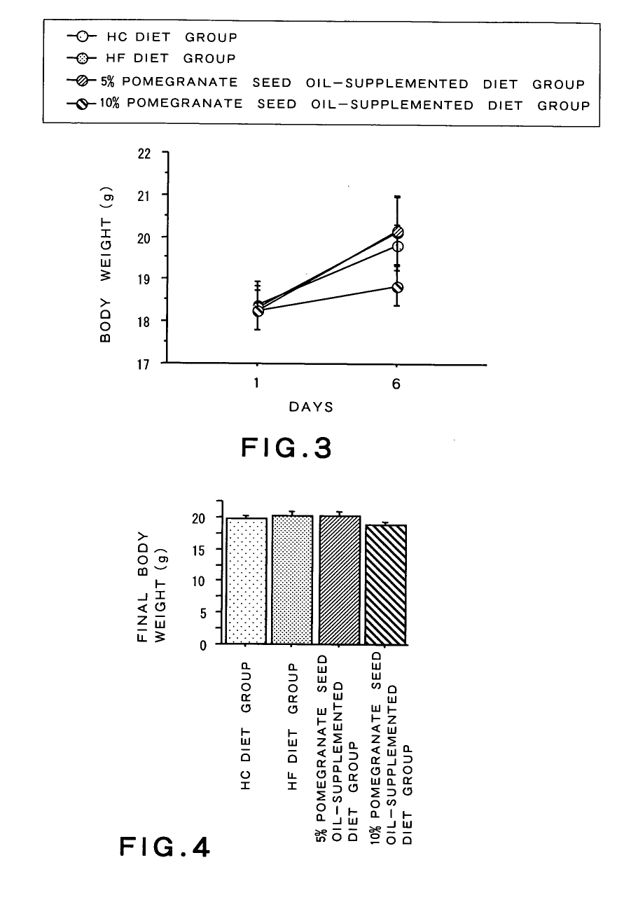

FIG. 3 shows the change in body weight in individual groups and FIG. 4 shows the final body weight in individual groups. No decrease in body weight was observed in the group fed the 5% pomegranate seed oil-supplemented diet; however, a decrease in body weight was observed in the group fed the 10% pomegranate seed oil-supplemented diet.

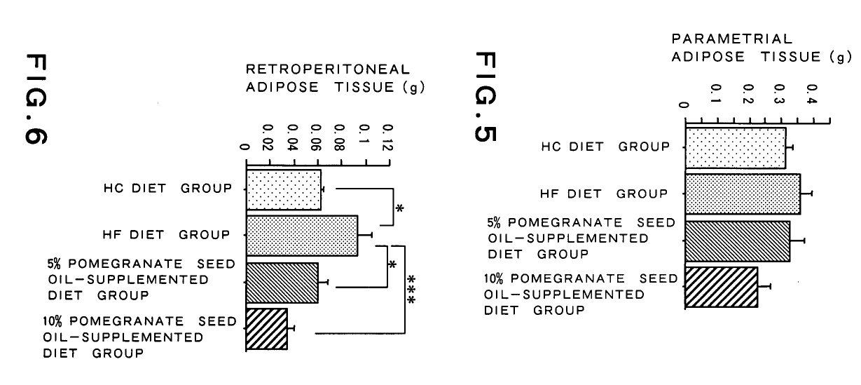

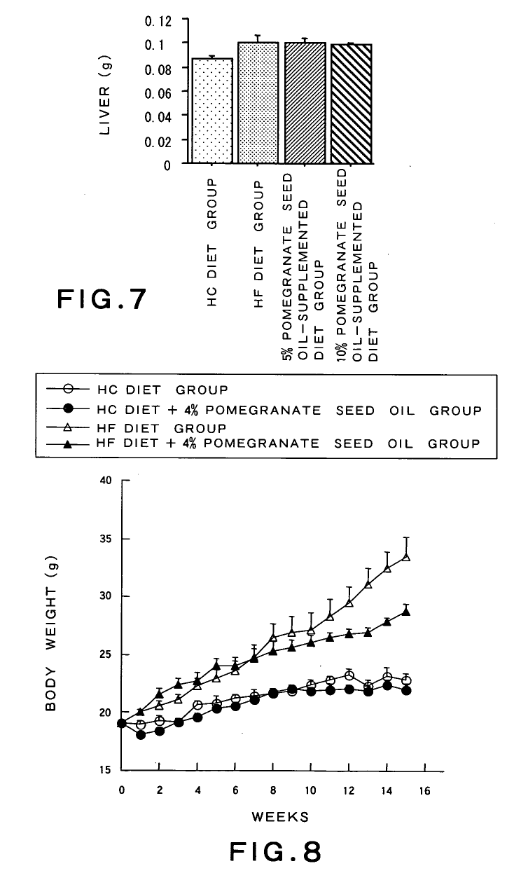

The weights of parametrial adipose tissue, retroperitoneal adipose tissue, and the liver are shown in FIGS. 5, 6, and 7. As for parametrial adipose tissue, a decrease depending on the concentration of pomegranate seed oil was observed. As for retroperitoneal adipose tissue, a significant decrease depending on the concentration of pomegranate seed oil was observed. As for the liver, no effect due to pomegranate seed oil intake was observed.

From the results above, it was suggested that pomegranate seed oil might control the accumulation of body fat for 6 days. However, since the amount of food intake was decreased in the group fed the 10% pomegranate seed oil-supplemented diet, the control of the body fat accumulation may be due to the decrease in the amount of food intake. Therefore, a long-term feeding using a sample supplemented with 5% or less pomegranate seed oil, which did not reduce the amount of food intake, was carried out to study the effect on lifestyle-related diseases.

### Example 2

### Effect of Long-Term Administration of Pomegranate Seed Oil on the Accumulation of Visceral Fat

### 1. Experimental Animals and Experimental Conditions

Female C57BL6j mice at 8 weeks of age (obtained from SLC) were divided into four groups of six animals: (1) a HC diet group, (2) a group fed a HC diet+4% pomegranate seed oil, (3) a HF diet group, and (4) a group fed a HF diet+4% pomegranate seed oil, and fed for 15 weeks. The animals were fed ad libitum during the feeding period.

The animals were sufficiently fed the day before dissection and the dissection was carried out under non-fasting conditions.

The compositions of individual samples administered are shown in Table 3.

TABLE 3

| | High carbohydrate diet | | | |
|---|---|---|---|---|
| | (g) | | High fat diet (g) | |
| | | +4% | | +4% |
| | | Pomegranate | | Pomegranate |
| | | seed oil | | seed oil |
| Safflower oil | 4.00 | | 33.5 | 29.50 |
| Pomegranate | | 4.00 | | 4.00 |
| seed oil | | | | |
| Casein | 20.00 | 20.00 | 29.00 | 29.00 |
| Sucrose | 11.03 | 11.03 | 23.29 | 23.29 |
| a-Starch | 55.17 | 55.17 | 0.00 | 0.00 |
| Vitamin mix | 1.00 | 1.00 | 1.45 | 1.45 |
| (AIN-93) | | | | |
| Mineral mix | 3.50 | 3.50 | 5.08 | 5.08 |
| (AIN-93) | | | | |
| Cellulose | 5.00 | 5.00 | 7.25 | 7.25 |
| L-cystine | 0.30 | 0.30 | 0.44 | 0.44 |
| Total | 100 | 100 | 100 | 100 |

### 2. Measurements

Body weight was measured daily during the feeding period.

The liver, parametrial adipose tissue, brown adipose tissue, retroperitoneal adipose tissue, subcutaneous adipose tissue, and total body fat were taken upon dissection, and weighed.

Body fat percentage was calculated from the final body weight and total body fat by dual energy X-ray absorptiometry (DEXA).

### 3. Experimental Results

### (1) HC Diet Group and Group Fed HC Diet+4% Pomegranate Seed Oil

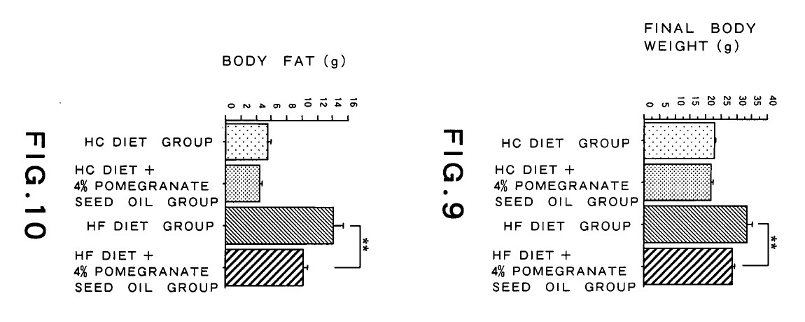

The change in body weight and the final body weight in individual groups are shown in FIG. 8 and FIG. 9, respectively. No significant difference in the change in body weight and the final body weight was observed between the groups.

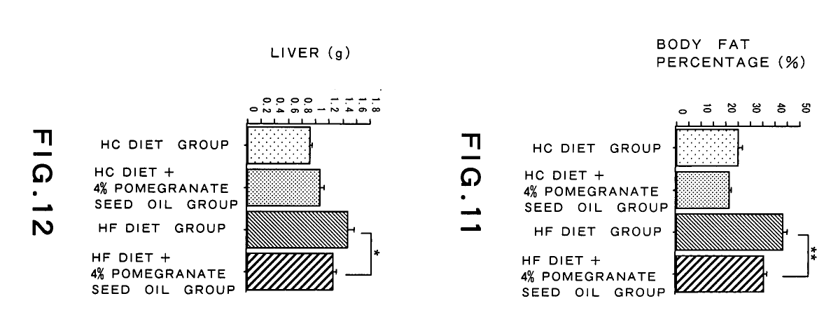

The weight of body fat and body fat percentage are shown in FIG. 10 and FIG. 11, respectively. Decreasing tendencies in the weight of body fat and the body fat percentage were observed in the group fed the pomegranate seed oil-supplemented diet.

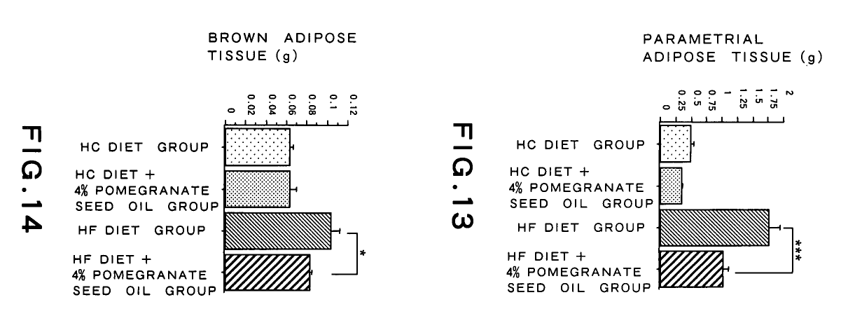

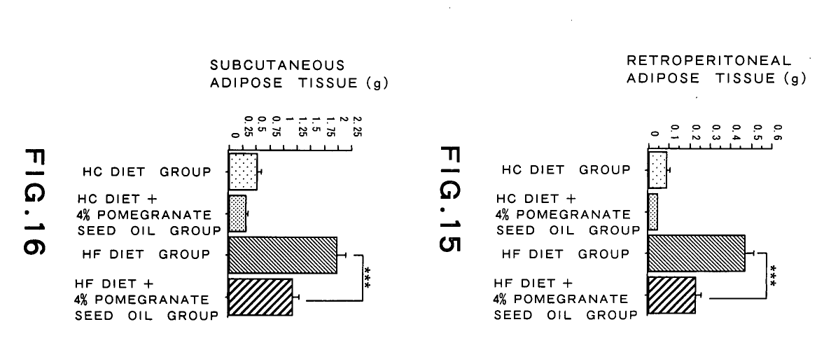

The weights of individual visceral fats in individual groups are shown in FIG. 12 to FIG. 15. In the group fed the pomegranate seed oil-supplemented diet, decreasing tendencies in the weights of parametrial adipose tissue and retroperitoneal adipose tissue were observed. In particular, retroperitoneal adipose tissue decreased by 52%.

The weight of subcutaneous fat in individual groups is shown in FIG. 16. A decreasing tendency in subcutaneous adipose tissue was observed in the group fed the pomegranate seed oil-supplemented diet.

Lipodystrophy-like symptoms, which are generated by the addition of conjugated linoleic acid, such as fatty liver, splenic hypertrophy, and disappearance of brown fat, were not observed.

From the results above, it has been recognized that pomegranate seed oil has an activity to reduce the weights of visceral fat and subcutaneous fat in a low fat diet. This suggests that the accumulation of visceral fat and subcutaneous fat may be improved by intake of pomegranate seed oil.

### (2) HF Diet Group and Group Fed HF Diet+4% Pomegranate Seed Oil

The change in body weight and the final body weight in individual groups are shown in FIG. 8 and FIG. 9, respectively. As for the body weight, a significant increase, which was observed in the HF diet group from 12 weeks after the start of experimental feeding, was controlled in the group fed the pomegranate seed oil-supplemented diet.

The weights of body fat and body fat percentage are shown in FIG. 10 and FIG. 11, respectively. The weight of body fat and the body fat percentage were significantly decreased in the group fed the pomegranate seed oil-supplemented diet.

The weights of individual visceral fats in individual groups are shown in FIG. 12 to FIG. 15. The weights of the liver, parametrial adipose tissue, and retroperitoneal adipose tissue were significantly decreased in the group fed the pomegranate seed oil-supplemented diet.

The weight of subcutaneous fat in individual groups is shown in FIG. 16. The weight of subcutaneous adipose tissue was significantly decreased in the group fed the pomegranate seed oil-supplemented diet.

Lipodystrophy-like symptoms (fatty liver, splenic hypertrophy, and disappearance of brown fat), which are generated by the addition of conjugated linoleic acid, were not observed. Further, a significant decrease in the weight of the liver and a significant decrease in the parametrial adipose tissue, which were not observed by the addition of conjugated linoleic acid, were observed. Furthermore, a partial decrease in adipose tissue, which is observed by the addition of conjugated linoleic acid, was not observed and all adipose tissue decreased to the same degree.

From the results above, it was recognized that pomegranate seed oil has an activity to reduce the weight of visceral fat and the weight of subcutaneous fat in a high fat diet. This suggests that the accumulation of visceral fat and subcutaneous fat may be improved by intake of pomegranate seed oil.

What is claimed is:

1. A method of treating diseases caused by the accumulation of body fat, comprising the step of administering a therapeutically effective amount of punicic acid or its ester to a mammal or fish.

2. The method according to claim 1, wherein the diseases caused by the accumulation of body fat are diseases caused by the accumulation of visceral fat or diseases caused by the accumulation of subcutaneous fat.

3. The method according to claim 2, wherein the diseases caused by the accumulation of visceral fat is fatty liver or visceral fat-type obesity.

4. The method according to claim 2, wherein the diseases caused by the accumulation of subcutaneous fat is subcutaneous fat-type obesity.

5. The method according to claim 1, wherein punicic acid is obtained from pomegranate seed oil.

6. The method according to claim 1, wherein the punicic acid content is 2.5 to 90% by weight.

7. A method of reducing the accumulation of body fat, comprising the step of administering an effective amount of punicic acid or its ester to a mammal or fish.

8. The method according to claim 7, wherein the body fat is visceral fat or subcutaneous fat.

9. The method according to claim 8, wherein the visceral fat is hepatic fat.
